(19) United States
(12) Patent Application Publication
(10) Pub. No.: US 2026/0328970 A1
(43) Pub. Date: Oct. 22, 2026

(54) METHOD AND APPARATUS FOR LASER PULSE COMPRESSION WITH DIFFRACTION GRATING PROTECTION FUNCTION

(71) Applicant: ELECTRONICS AND TELECOMMUNICATIONS RESEARCH INSTITUTE, Daejeon (KR)

(72) Inventors: Dongho SHIN, Daejeon (KR); Eunsol KO, Daejeon (KR); Hyunwoo SONG, Daejeon (KR); Euisang YU, Daejeon (KR)

(21) Appl. No.: 19/652,951

(22) Filed: Apr. 20, 2026

(30) Foreign Application Priority Data
Apr. 18, 2025 (KR) 10-2025-0051126
Apr. 2, 2026 (KR) 10-2026-0059873

(51) Int. Cl. G02B 1/14; A61N 5/10; G02B 19/00

(52) U.S. Cl. CPC G02B 1/14; A61N 5/1077; G02B 19/0019; A61N 2005/1088

(57) ABSTRACT

A laser pulse compressor includes: a diffraction grating for compressing an incident laser pulse; a nozzle spraying a protective substance such that the protective substance is applied onto a surface of the diffraction grating after compression of each laser pulse group is completed; and a light source configured to irradiate a removal light beam for removing the protective substance toward the surface of the diffraction grating after application of the protective substance is completed and before resumption of compression of a subsequent laser pulse group.

### CROSS-REFERENCE TO RELATED APPLICATION

This application claims priority under 35 U.S.C § 119 to Korean Patent Application No. 10-2025-0051126 filed in the Korean Intellectual Property Office on Apr. 18, 2025, and Korean Patent Application No. 10-2026-0059873 filed in the Korean Intellectual Property Office on Apr. 2, 2026, the entire contents of which are hereby incorporated by reference.

### TECHNICAL FIELD

The present disclosure relates to an apparatus and method for compressing a laser pulse including a diffraction grating protection function, and more particularly, to a technology for maintaining constant performance of a laser by preventing surface contamination of a diffraction grating inside a laser pulse compressor.

### BACKGROUND OF THE INVENTION

Recently, a particle beam accelerator using a high-power femtosecond laser is being developed as a means capable of replacing a synchrotron radiation accelerator. Specifically, when a laser pulse focused at an intensity of 10²⁰ W/cm² or higher is irradiated onto a solid target, particles such as protons and heavy ions are emitted with high energy, and efforts are underway to commercialize devices that utilize this phenomenon for cancer treatment. In order for such a laser pulse-based cancer treatment device to be commercialized, an accelerated particle beam needs to have a constant energy, a spatial distribution, and a sufficient flux. Furthermore, to achieve this, basic characteristics of the laser such as a pulse width, a pulse contrast ratio, a beam shape, and beam pointing are typically maintained at a same value at all times. In other words, there is a demand for a laser apparatus capable of maintaining physical characteristics identically for a long period of time and securing constant performance.

Meanwhile, a high-power laser apparatus includes a laser pulse compressor compressing a laser pulse to amplify energy, and component damage caused by a high-power laser frequently occurs inside the laser pulse compressor. In particular, as laser pulse energy is intensively irradiated onto a diffraction grating in the laser pulse compressor, a large amount of energy is applied to a surface of the diffraction grating, and thus a possibility of surface contamination or damage of the diffraction grating caused by an interaction between trace molecules remaining inside a vacuum chamber and a high-energy laser pulse is high. In this case, diffraction efficiency of the diffraction grating rapidly degrades, causing a problem of non-uniformity and degradation of performance of the laser apparatus.

In order to prevent such contamination and damage of the diffraction grating, an operation of the laser apparatus needs to be stopped, a vacuum state of the laser pulse compressor needs to be broken, and then the diffraction grating needs to be cleaned. However, according to such a protection method of the diffraction grating, a considerable amount of time is required for maintenance of the laser apparatus, and a limitation exists in that a situation of inevitably stopping an operation of the laser apparatus occurs in an environment where continuous operation of the laser apparatus is essential such as in a hospital.

### PRIOR ART DOCUMENTS

(Patent Document 1) Korean Patent Application Publication No. 10-2745394 (Published on Dec. 17, 2024)

### SUMMARY OF THE INVENTION

An objective of the present disclosure includes fundamentally preventing contaminants from being adsorbed to a surface of a diffraction grating when a laser pulse compressor is not operating.

Furthermore, the above-described objective includes preventing contamination of the diffraction grating by minimizing a time interval from a time point when compression of a laser pulse is completed to a time point when a protective substance is applied to the surface of the diffraction grating.

Furthermore, the above-described objective includes preventing degradation in performance of the laser pulse compressor and minimizing a maintenance work of a laser apparatus by maintaining diffraction efficiency at a constant level by preventing contamination of the diffraction grating.

Furthermore, the above-described objective includes allowing application apparatuses such as an ultra-high dose rate particle beam generator and a cancer treatment device utilizing an application apparatus to stably operate for a long period of time without stopping an operation by stabilizing laser pulse characteristics of the laser pulse compressor for a long period of time.

However, the objective of the present disclosure is not limited to those mentioned above, and another objective not mentioned will be clearly understood by those of ordinary skill in the art to which the present disclosure pertains from the following description.

In accordance with a first aspect of the present disclosure, there is provided a laser pulse compressor including: a diffraction grating for compressing an incident laser pulse; a nozzle spraying a protective substance such that the protective substance is applied onto a surface of the diffraction grating after compression of each laser pulse group is completed; and a light source configured to irradiate a removal light beam for removing the protective substance toward the surface of the diffraction grating after application of the protective substance is completed and before resumption of compression of a subsequent laser pulse group.

Further, the surface may be activated to a predetermined energy state after completion of the compression of each laser pulse group, and the protective substance may be applied onto the surface of the diffraction grating in an activated state.

Further, the protective substance may include an organic material having a thiol group.

Further, the protective substance may form a protective layer adhered to the surface of the diffraction grating by a chemical bond to block contact with contaminants.

Further, the nozzle may be disposed such that a spray direction of the protective substance directs an area onto which the laser pulse is irradiated among the surface of the diffraction grating.

Further, the nozzle may be disposed such that an irradiation area of the laser pulse is 80% or less of an area where the protective substance is applied.

Further, the nozzle may be driven in response to a signal indicating completion of the compression of each laser pulse group.

Further, the nozzle may be driven by a predetermined driving signal, and the driving signal may be generated using the signal indicating the completion of the compression.

Further, a spray start time of the protective substance from the nozzle may be determined based on a time length during which each laser pulse group is irradiated onto the diffraction grating.

Further, the spray start time of the nozzle may be advanced as an irradiation time of each laser pulse group increases.

Further, an irradiation time length of the removal light beam of the protective substance may be determined depending on a time length of spraying the protective substance.

Further, as the spray time of the protective substance increases, the irradiation time length of the removal light beam for removing the protective substance increases.

Further, at least one of a completion time of the compression of each laser pulse group and a spray time of the protective substance may be determined based on a pre-trained artificial intelligence model.

In accordance with a second aspect of the present disclosure, there is provided a method for protecting a diffraction grating of a laser pulse compressor performed by the laser pulse compressor, the laser pulse compressor including the diffraction grating, the method including: spraying a protective substance toward a surface of the diffraction grating to apply the protective substance to the surface of the diffraction grating after compression of each laser pulse group by the diffraction grating is completed; and irradiating a removal light beam for removing the protective substance toward the surface of the diffraction grating after application of the protective substance is completed and before resumption of the compression of a subsequent laser pulse group.

Further, the protective substance may include an organic material having a thiol group.

Further, the protective substance may form a protective layer adhered to the surface of the diffraction grating by a chemical bond to block contact with contaminants.

Further, the laser pulse compressor may include a nozzle, and the method may further include: driving the nozzle in response to a signal indicating completion of the compression of each laser pulse group.

Further, the nozzle may be driven by a driving signal generated from the signal indicating the completion of the compression.

Further, at least one of a completion time of the compression of each laser pulse group and a spray time of the protective substance may be determined based on a pre-trained artificial intelligence model.

In accordance with a third aspect of the present disclosure, there is provided a particle beam therapy apparatus including: the laser pulse compressor described above; a focusing optical system focusing a compressed laser pulse output from the laser pulse compressor; a target generating therapeutic particles using the compressed laser pulse focused by the focusing optical system; and a particle beam provider configured to provide a therapeutic particle beam using the therapeutic particles generated by the target.

According to an aspect of the present disclosure, surface contamination of the diffraction grating occurring in a period from after an operation of the laser pulse compressor is stopped to before the operation is resumed is prevented or suppressed.

Furthermore, a variation in a state (e.g., contamination or damage) of the surface of the diffraction grating is mitigated while a vacuum environment is maintained, so that a degradation in diffraction efficiency of the diffraction grating is mitigated and performance or an output of the laser apparatus is consistently maintained.

Furthermore, a deviation in a surface state of the diffraction grating between operation cycles of the laser pulse compressor is reduced, and accordingly, a variation in a characteristic of laser pulse compression is reduced. Therefore, a maintenance burden related to inspection, cleaning, or part replacement of the laser apparatus is reduced.

Furthermore, when applied to an application apparatus including the laser pulse compressor according to the present disclosure, operational stability of the application apparatus is improved under a condition where a variation in the characteristic of the laser pulse compression is reduced.

Meanwhile, a technology related to application and removal of the protective substance of the present disclosure is applied to a surface treatment process other than protecting the surface of the diffraction grating.

The effects obtainable from the present disclosure are not limited to the effects mentioned above, and other effects not mentioned will be clearly understood by those of ordinary skill in the art to which the present disclosure pertains from the following description.

### BRIEF DESCRIPTION OF THE DRAWINGS

FIG. 1 is a schematic diagram illustrating an entire system of a laser apparatus.

FIG. 2 is a schematic diagram illustrating an internal configuration of a laser pulse compressor.

FIG. 3 is a functional block diagram illustrating an internal configuration and an operation of a laser pulse compressor.

FIG. 4 is a flowchart illustrating a method for protecting a diffraction grating of a laser pulse compressor performed by the laser pulse compressor.

FIG. 5 is a schematic diagram illustrating a configuration of a particle beam therapy apparatus.

### DETAILED DESCRIPTION OF THE INVENTION

The present disclosure is not limited to the embodiments disclosed below but may be implemented in various different forms, and the present embodiments are merely provided to complete the disclosure of the present disclosure and to fully inform those of ordinary skill in the art to which the present disclosure pertains of the scope of the disclosure, and the present disclosure is defined by the scope of the claims. In describing the embodiments of the present disclosure, when it is determined that a detailed description of a known function or configuration may unnecessarily obscure the gist of the present disclosure, the detailed description thereof will be omitted. In addition, terms to be described later are terms defined in consideration of functions in the embodiments of the present disclosure, varying according to intentions or customs of users and those skilled in the art. Therefore, the definition thereof should be made based on contents throughout the present specification.

First, terms used in the present specification will be briefly described, and the present disclosure will be described in detail. The terms used in the present specification are selected from general terms currently widely used as much as possible while considering the functions of the present disclosure, varying according to intentions or precedents of technicians working in the field, emergence of new technologies, and the like. Furthermore, in specific cases, there are terms arbitrarily selected by an applicant, and in this case, meanings thereof will be described in detail in a description part of the corresponding disclosure. Therefore, the terms used in the present disclosure should be defined based on the meanings of the terms and the contents throughout the present disclosure, rather than simple names of the terms. Throughout the specification, when a certain part is said to “include” a certain component, it means that other components may be further included rather than excluding other components unless specifically stated to the contrary.

Furthermore, in the present disclosure, the term ‘unit’ refers to a component performing a specific function or operation, and the corresponding component may be implemented as hardware, software, or a combination thereof. Furthermore, the ‘unit’ may be implemented by being separated into or combined with one or more components.

Hereinafter, embodiments of the present disclosure will be described in detail with reference to the accompanying drawings so as to be easily carried out by those of ordinary skill in the art to which the present disclosure pertains.

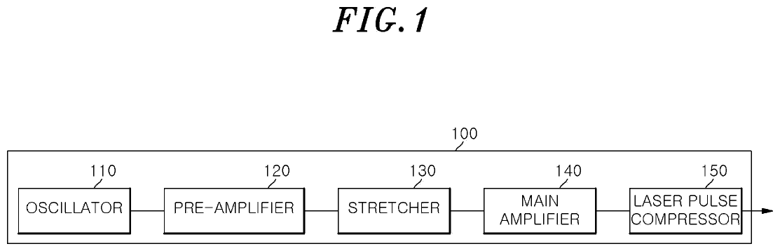

FIG. 1 is a schematic diagram illustrating an entire system of a laser apparatus.

Referring to FIG. 1, a laser apparatus 100 for implementing an ultra-short pulse and an ultra-high power in units of femtoseconds includes an oscillator 110, a pre-amplifier 120, a stretcher 130, a main amplifier 140, and a laser pulse compressor 150.

In one embodiment, the laser apparatus 100 may further include a shutter for selectively blocking a laser pulse from being incident on the laser pulse compressor 150.

Specifically, the oscillator 110 generates a seed laser pulse, and energy of the generated laser pulse is amplified while passing through the pre-amplifier 120 and the main amplifier 140. Further, in order to prevent optical components included in the laser apparatus 100 from being damaged by the laser pulse, the laser apparatus 100 temporally stretches a width of the laser pulse through the stretcher 130, and thereafter, by re-compressing the width of the stretched laser pulse through the laser pulse compressor 150, ultra-high power of a terawatt (TW) or petawatt (PW) class may be achieved.

The laser pulse compressor 150 according to the embodiment of the present disclosure corresponds to the laser pulse compressor 150 as an optical component operating in an operation of re-compressing the width of the stretched laser pulse as a final output operation among the entire system of the laser apparatus 100.

A diffraction grating for compressing the laser pulse is disposed inside the laser pulse compressor 150, and the laser pulse is compressed through the diffraction grating.

During this process, as a high-energy laser pulse is irradiated onto a surface of the diffraction grating, a phenomenon in which a surface material (e.g., gold (Au)) of the diffraction grating undergoes activation may occur.

Further, an activated surface of the diffraction grating has high reactivity with organic molecules or moisture remaining in a vacuum chamber, and thereafter, while an operation of the laser pulse compressor 150 is terminated and the laser pulse is not irradiated onto the diffraction grating, the surface material of the diffraction grating binds with the organic molecules or moisture, and accordingly, surface contamination and damage of the diffraction grating and a degradation in diffraction efficiency caused thereby may be caused.

Therefore, an objective of the laser pulse compressor 150 according to the embodiment of the present disclosure is to prevent contamination and damage of the surface of the diffraction grating by applying a protective substance to the surface of the diffraction grating to protect the diffraction grating and removing the applied protective substance before the laser pulse compressor 150 operates again thereafter.

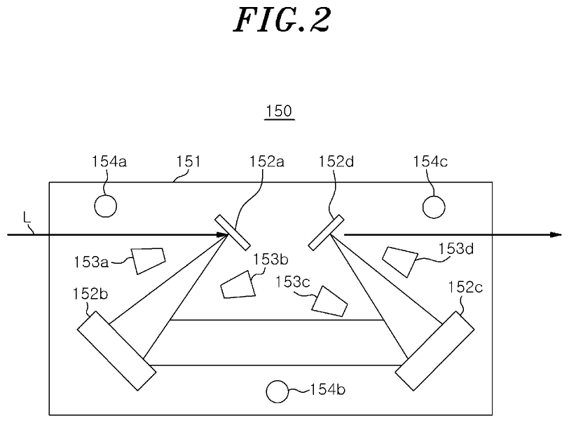

FIG. 2 is a schematic diagram illustrating an internal configuration of the laser pulse compressor 150.

Referring to FIG. 2, the laser pulse compressor 150 has a physical configuration including a diffraction grating 152 disposed inside a vacuum chamber 151, a nozzle 153 spraying a protective substance onto a surface of the diffraction grating, and a light source 154 irradiating a light beam onto the surface of the diffraction grating to remove the applied protective substance.

Specifically, looking at a propagation path of the laser pulse inside the chamber, a laser pulse incident from an outside is diffracted and dispersed by a first diffraction grating 152a, spatially or temporally compressed while passing through a second diffraction grating 152b and a third diffraction grating 152c, and may form a main propagation path of the laser pulse in a form finally output through a fourth diffraction grating 152d. In a process where the laser pulse sequentially passes through the plurality of diffraction gratings 152a to 152d as described above, a pulse width is extremely compressed, and in particular, as at least one of the plurality of diffraction gratings is disposed at a rear stage, an energy density applied to a surface sharply increases.

Accordingly, the laser pulse compressor 150 may include nozzles 153a to 153d and light sources 154a to 154c arranged at a periphery thereof as components to protect the diffraction gratings 152a to 152d exposed to the high-energy laser pulse. As shown in the drawings, the nozzles 153a to 153d may be spaced apart and disposed in a free space inside the chamber so as not to overlap (so as not to physically block or interfere) with the main propagation path of the laser pulse.

Further, the nozzles 153a to 153d may be tilted and fixed at a specific angle to be directed toward surfaces of the diffraction gratings 152a to 152d targeted by the laser pulse.

In one embodiment, disposition positions and spray angles of the nozzles 153a to 153d may be disposed such that an application area formed by the protective substance sprayed from the nozzles 153a to 153d reaching the surface of the diffraction gratings 152a to 152d entirely covers an area where the laser pulse is irradiated onto the surface. Furthermore, in one embodiment, the nozzles 153a to 153d may be disposed such that an irradiation area of the laser pulse is 80% or less of an area where the protective substance is applied.

Furthermore, the light sources 154a to 154c are spaced apart and disposed inside the vacuum chamber 151 so as not to overlap with the propagation path of the laser pulse, and may be disposed at a position and a distance capable of transmitting uniform optical energy to entire surfaces of the diffraction gratings 152a to 152d to effectively vaporize the applied protective substance.

Next, a control system for protecting the diffraction grating of the laser pulse compressor 150 will be described.

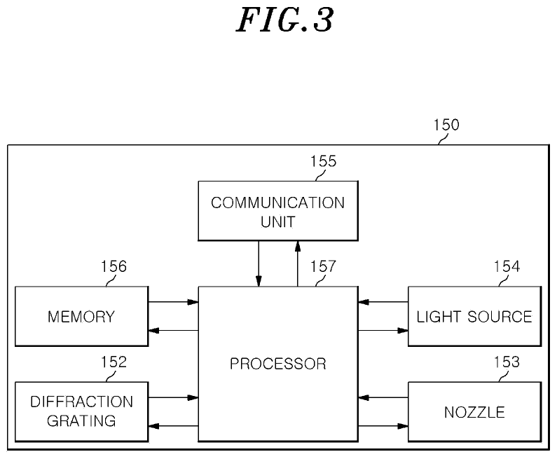

FIG. 3 is a functional block diagram illustrating an internal configuration and an operation of the laser pulse compressor 150.

In one embodiment, the laser pulse compressor 150 may include a communication unit 155, a memory 156, a processor 157, a diffraction grating 152, a nozzle 153, and a light source 154. However, the spirit of the present disclosure is not limited by components illustrated in FIG. 3, and an apparatus according to the embodiment of the present disclosure may be implemented including more or fewer components than illustrated. Furthermore, each component may be provided in plurality.

First, the communication unit 155 may include one or more modules arranged to exchange a wired or wireless communication or an electrical signal between the laser pulse compressor 150 and an external apparatus such as a main controller of the laser apparatus 100, e.g., a wired or wireless communication module. Specifically, the wired communication module may include Ethernet, USB communication, optical cable communication, and the like, and the wireless communication module may include Wi-Fi, Bluetooth, a mobile communication network (LTE, 5G, etc.). In the present disclosure, the laser pulse compressor may receive a high-speed trigger signal (e.g., a TTL (Transistor-Transistor Logic) signal, etc.) for controlling an output of the laser pulse from the outside in real time through the communication unit 155.

Next, the memory 156 stores various types of data and algorithms for an operation of the laser pulse compressor. For example, information such as a received laser control signal history, a protective substance spray timing, an irradiation time of the light source 154 may be temporarily or permanently stored in the memory 156. Such a memory 156 may include at least one type of physical storage medium among a flash memory, a hard disk, an SSD, a RAM, an SRAM, a ROM, and an EEPROM, and further, may operate in a form of a cloud server performing a storage function on the Internet.

Next, the processor 157 typically controls an overall operation of the laser pulse compressor 150 by executing at least one instruction stored in the memory 156. That is, the processor 157 electronically directs valve opening/closing of the nozzle 153, lighting of the light source 154, and fine angle adjustment (actuator driving) of the diffraction grating 152. Such a processor 157 may be implemented using at least one among a central processing unit (CPU), a micro controller unit (MCU), ASICs, DSPs, and FPGAs.

Furthermore, according to the embodiment of the present disclosure, the processor 157 may operate based on an artificial intelligence (AI) model or a machine learning algorithm. For example, the processor 157 learns data such as an irradiation energy pattern of a past laser pulse, a change in temperature/vacuum level in the chamber using a neural network model (e.g., DNN, CNN, RNN, etc.), and based on this, may predict and control an optimum spray amount of the nozzle 153 or an optimum ultraviolet irradiation time of the light source 154 in real time.

Finally, the diffraction grating 152, the nozzle 153, and the light source 154 are components performing a physical/optical operation according to a control signal of the processor 157, and geometrically disposed inside the chamber to perform contamination prevention and a restoration mechanism (removal of the protective substance) of the surface of the diffraction grating as described above with reference to FIG. 2.

Hereinafter, based on an understanding of the laser pulse compressor 150 reviewed above, a specific configuration and a function or operation of the laser pulse compressor will be described.

However, in the present disclosure, what is described as being performed by the laser pulse compressor may be understood to be performed by the processor 157 executing at least one instruction stored in the memory 156.

First, the diffraction grating 152 disposed inside the laser pulse compressor 150 is used for compression of an incident laser pulse.

Here, the diffraction grating 152 may mean an optical element diffracting and dispersing a laser pulse by a periodic structure being formed on a surface on which the laser pulse is incident.

Furthermore, the diffraction grating 152 may be disposed on a propagation path of the laser pulse along which the laser pulse propagates inside the laser pulse compressor 150, and an area onto which the laser pulse is irradiated may be formed on a surface of the diffraction grating 152.

In one embodiment, the diffraction grating 152 may include a form in which an outermost layer thereof is coated with a gold (Au) thin film so that an interaction with a protective substance described later occurs.

For example, when the protective substance includes a functional group containing sulfur (S) (e.g., a thiol group (—SH)), a gold-sulfur (Au—S) bond may be formed between the gold (Au) surface and the protective material. However, the material or structure of the surface layer of the diffraction grating 152 is not limited thereto, and may be implemented in various forms to enable bonding or adsorption with the protective material.

Furthermore, a periodic groove structure may be formed on the surface of the diffraction grating 152, and the groove structure may be a microstructure related to an optical function of the diffraction grating 152.

In one embodiment, as the laser pulse is irradiated onto the surface of the diffraction grating 152, the surface may be activated to a predefined energy state after completion of the compression of the laser pulse.

Here, “activation” may refer to a change in the state of the surface after laser pulse irradiation, such that the surface transitions into a condition in which bonding or adsorption with molecular species (e.g., organic components and moisture) that may be present in the device is more likely to occur.

Furthermore, in one embodiment, the protective substance to be described later may be applied to the surface in the activated state. In this regard, a specific composition and application method of the protective substance and characteristics of a layer formed on the surface by the protective substance will be described later.

According to the diffraction grating 152 and surface characteristics thereof as described above, a variation in a surface state of the diffraction grating 152 occurring during an operation process of the laser pulse compressor 150 may be reduced, and progress of surface contamination may be suppressed, mitigating a degradation in diffraction efficiency.

Furthermore, the tendency of contaminants to remain or accumulate on a surface having a microstructure can be reduced, and changes in the surface state from the end of one operation to the start of the next operation can also be minimized.

Next, the nozzle 153 of the laser pulse compressor 150 sprays the protective substance to apply the protective substance to the surface of the diffraction grating 152 after the compression of the laser pulse is completed.

Here, the nozzle 153 means a configuration for spraying a fluid (e.g., protective substance) toward the surface of the diffraction grating 152. For example, the nozzle 153 may be fluidically connected to a reservoir (not shown) in which the protective substance is stored or to a supply line, and the start and stop of spraying of the protective substance may be controlled through a valve (e.g., a solenoid valve) or a flow rate control unit.

For example, the nozzle 153 may be implemented in a form in which the protective substance is sprayed by the valve opening after the completion of the compression, and the spraying is terminated by the valve closing when a predetermined spray time elapses or a predetermined spray amount is reached. Further, the predetermined spray time or the predetermined spray amount may be set to be a state in which the protective substance is applied to the surface of the diffraction grating 152 (e.g., forming an application area).

The protective substance may be a material for suppressing a variation in a surface state caused by contact or adsorption of contaminants on the surface of the diffraction grating 152.

The protective substance may include an organic material having a thiol group (—SH group).

For example, the protective substance may be provided in a form of a solution in which an organic compound containing a thiol group is dissolved in a solvent or in a vaporized and sprayable form.

Furthermore, the protective substance is adsorbed to the surface of the diffraction grating 152 by a chemical bond, forming a protective layer blocking contact with contaminants. For example, when an outermost layer of the surface of the diffraction grating 152 is gold (Au), it may be implemented in a form in which a protective layer is formed as a gold-sulfur (Au—S) bond is formed between a protective substance containing a thiol group and the gold surface. However, the manner in which the protective layer is formed may vary depending on the surface condition, spraying conditions, and the composition of the protective substance, and the like.

The nozzle 153 may be disposed such that a spray direction of the protective substance directs an area onto which the laser pulse is irradiated among the surface of the diffraction grating 152. For example, the nozzle 153 may be obliquely disposed to direct the surface of the diffraction grating 152, and a position and a spray angle of the nozzle 153 may be set such that the spray direction includes a laser irradiation area.

Further, the nozzle 153 may further include an adjustment unit configured to adjust a position and a spray angle of the nozzle 153. The adjustment unit may include a rotation structure and/or a sliding structure for changing an inclination and a direction of the nozzle 153, thereby enabling precise control of a spraying region of the protective material in response to laser irradiation conditions or a position change of the diffraction grating 152. Furthermore, the adjustment unit may be operated manually or automatically, and in the case of automatic operation, the adjustment unit may be configured to be interlocked with the processor 157 so that the position and the spray angle of the nozzle 153 are variably adjusted according to a laser irradiation position.

Furthermore, the nozzle 153 may be disposed such that an irradiation area of the laser pulse is 80% or less of an area where the protective substance is applied. For example, by combining a spray pattern (e.g., spray angle range) of the nozzle 153, a distance to the surface of the diffraction grating 152, and an arrangement position of the nozzle 153, a protective substance application area may be set to include the laser irradiation area and make the irradiation area 80% or less of the application area.

Meanwhile, the completion of the compression of the laser pulse means transitioning from a state in which the laser pulse is irradiated onto the diffraction grating 152 to a state in which the laser pulse is not irradiated, and may be implemented in various manners such as a manner in which generation or output of the laser pulse is stopped, a manner in which the laser pulse is physically blocked, or a combination thereof.

The laser apparatus 100 may include a shutter disposed inside or outside the laser pulse compressor 150 on a propagation path of the laser pulse. The shutter may be a mechanical shutter driven to physically block the laser pulse.

Next, the nozzle 153 may be driven in response to a signal indicating completion of the compression of the laser pulse, or may be driven by a predefined driving signal.

The signal indicating the completion of the compression of the laser pulse may include a signal generated according to stopping of generation or output of the laser pulse of the laser apparatus 100, or a signal generated based on a driving state of the shutter.

For example, the signal indicating the completion of the compression of the laser pulse may include an electrical signal (e.g., TTL level signal) output from the laser apparatus 100.

Furthermore, for example, the signal indicating the completion of the compression of the laser pulse is a signal generated based on a driving state of the shutter, and may include a driving signal of the shutter, or a signal generated by detecting a state in which the shutter is completely closed. Accordingly, driving of the nozzle 153 may be initiated after the shutter is completely closed.

Meanwhile, when the nozzle 153 is driven by a predetermined driving signal, the driving signal may be implemented in at least one of (i) a manner generated from a signal indicating completion of compression and (ii) a manner generated by detecting a change in a signal indicating completion of compression.

For example, the (i) manner may be a form in which the nozzle is driven using an inverter circuit converting a logic level of a compression completion signal to match a driving level of the nozzle 153, and the (ii) manner may be a form in which the nozzle is driven using a trigger circuit generating a pulse-type driving signal used for driving the nozzle by detecting an edge (rising or falling) of a compression completion signal. However, the inverter circuit and the trigger circuit are exemplary, and generation of the nozzle driving signal may be implemented by various signal processing configurations performing at least one function among level conversion, change detection, and pulse-type driving signal generation.

A spray start time of the nozzle 153 may be determined based on a length of time during which the laser pulse is irradiated onto the diffraction grating 152. For example, the processor 157 may select a delay time for driving the nozzle 153 according to an irradiation time length of the laser pulse from a preset table or calculate and apply a delay time according to a laser operation condition. Furthermore, a spray start time may be set earlier as an irradiation time length of the laser pulse is longer. For example, a plurality of recipes (operation rules) may be set such that a delay time decreases as an irradiation time length increases.

According to the nozzle 153 and the protective substance as described above, contact or adsorption of contaminants on the surface of the diffraction grating 152 may be suppressed after termination of an operation of compressing a laser pulse, and a variation in a surface state of the diffraction grating 152 may be reduced. Furthermore, when an application area is set to include a laser irradiation area, a change in a surface state in the laser irradiation area may be mitigated, and a change in a surface state between operation cycles may be reduced.

Next, the light source 154 of the laser pulse compressor 150 irradiates a removal light beam of the protective substance toward the surface of the diffraction grating 152 after application of the protective substance is completed and before resumption of the laser pulse compression.

Here, the light source 154 may mean a component emitting a light beam for removing the protective substance applied to the surface of the diffraction grating 152, and the removal light beam may include ultraviolet rays.

Furthermore, meanings of completion of application and resumption of compression may vary according to embodiments, and for example, the completion of application may be defined by an elapse of a predetermined spray time or reaching a predetermined spray amount, and the resumption of compression may be defined by a time point at which a laser pulse is re-incident into the laser pulse compressor 150 or a compression operation is initiated.

The light source 154 may be disposed to directly irradiate a removal light beam of the protective substance toward the surface of the diffraction grating 152, and an irradiation direction of the light beam, an irradiation range, and a relative position between the light source 154 and the surface may be set to remove a protective layer formed on the surface of the diffraction grating 152. For example, the light source 154 may be disposed at a position not directly interfering with a path of the laser pulse inside the chamber while being disposed such that a removal light beam is irradiated toward the surface of the diffraction grating 152.

The removal light beam may be irradiated in a continuous wave (CW) mode. For example, when a continuous wave light source 154 is used, an irradiation condition of a removal light beam may be set primarily based on an irradiation time length, and the irradiation time length may be determined according to a correspondence relation with a spray condition described later.

Furthermore, the removal light beam may be irradiated in a pulse mode. For example, when a pulsed light source 154 is used, an irradiation condition of a removal light beam may be set based on a cumulative irradiation amount (energy density) or the number of pulses/repetition rate, and may be operated to perform removal of a protective substance according to at least one criterion of an irradiation time length and an irradiation amount. Here, an irradiation amount may be defined as a product of an irradiation intensity and an irradiation time in the case of continuous wave, and may be defined based on a product of energy per pulse and the number of pulses in the case of the pulsed light source 154.

An irradiation time length of a removal light beam of a protective substance may be determined depending on a time length of spraying the protective substance.

Furthermore, an irradiation time length of a removal light beam of a protective substance may be set longer as a spray time of the protective substance is longer. Setting of such an irradiation condition may be set to be changeable according to an operation condition.

Furthermore, in order to increase removal efficiency of a protective substance, a removal light beam may be irradiated in a state in which a vacuum level of the vacuum chamber 151 is adjusted. For example, the vacuum chamber 151 is maintained in a high vacuum state (e.g., 10⁻⁶ torr or less) while compression of a laser pulse is performed, and after application of a protective substance is completed, a vacuum level of the vacuum chamber 151 may be adjusted to a 10⁻³ torr level according to a type of the protective substance and an interaction mechanism with a removal light beam. In this case, a removal light beam is irradiated after isolating the vacuum chamber 151 by closing a gate valve connected to the vacuum chamber 151 (a valve disposed between the vacuum chamber 151 and an external vacuum exhaust line to selectively isolate the vacuum chamber 151), and after removal of the protective substance is completed, irradiation of the removal light beam is stopped and the gate valve is opened to return a vacuum level of the vacuum chamber 151 to a high vacuum state.

According to such an operation of the light source 154 and an operating environment, remaining of a protective layer formable on the surface of the diffraction grating 152 may be reduced, and a variation in a surface state of the diffraction grating 152 between operation cycles may be mitigated. Furthermore, a difference in a state of the surface of the diffraction grating 152 at a time of resumption of operation may be reduced, and thereby, a variation in output characteristics according to an operating condition of the laser pulse compressor 150 may be mitigated, allowing consistency of performance of the laser pulse compressor 150 to be maintained.

The light source 154 may be configured as a single piece, or when a plurality of diffraction gratings 152a to 152d are disposed, a plurality of light sources may be disposed to correspond to each of the diffraction gratings 152a to 152d.

Furthermore, a correspondence relationship between the light source 154 and the diffraction gratings 152a to 152d may be set in consideration of a direction of a periodic structure of a surface of a diffraction grating, and one light source 154 may irradiate a removal light beam to a plurality of diffraction gratings, or a plurality of light sources 154 may cooperate to irradiate a removal light beam to one diffraction grating.

Next, at least one of a completion time of compressing a laser pulse and a spray time of a protective substance may be determined using a pre-trained artificial intelligence model.

An embodiment may include an operation of predicting or estimating a completion time of compression of a laser pulse or a time of occurrence of a corresponding event, thereby setting a spray start time of a protective substance to be close to the completion time of compression.

The laser pulse compressor 150 may include the memory 156 storing a pre-trained artificial intelligence model and at least one instruction, and the processor 157 executing the instruction to use the artificial intelligence model.

The artificial intelligence model may predict a completion time of compression based on at least one input data indicating a laser operation state, and the input data may include at least one of an operation sequence state, a repetition rate or pulse count, a shutter state, and a compression completion-related signal.

The processor 157 may determine a spray start time of the nozzle 153 based on the predicted compression completion time, and may set a generation time of a driving signal of the nozzle 153 or a delay time for driving the nozzle 153 such that the spray start time is close to the compression completion time.

In addition to determining the spray start time of the nozzle 153 based on the predicted compression completion time, the processor 157 may set at least one of a spray time length and a spray amount for the nozzle 153 to spray a protective substance. For example, the processor 157 may select an application time length or a spray amount of a protective substance as at least one of a plurality of recipes or calculate and apply the same according to a preset rule or a table based on input data on a laser operation state or a surface state of the diffraction grating 152.

Furthermore, the processor 157 may set at least one of an irradiation time length of a removal light beam and an irradiation condition of the light source 154 in conjunction with at least one of a spray time length and a spray amount of the nozzle 153. For example, when a spray time length of a protective substance is set long, an operation rule causing an irradiation time length of a removal light beam to be set long may be applied, and the irradiation time length may be defined in a form of a rule, a table, or a function and updated according to an operation condition.

According to such AI model-based operation, a time difference between the end of laser pulse compression and the start of spraying of the protective substance can be reduced, and variations in the surface state of the diffraction grating 152 from the end of one operation to the start of the next operation can be mitigated. In addition, even when operating conditions change, variations in the spraying timing can be reduced, thereby minimizing changes in the surface state between operation cycles.

Meanwhile, in the present disclosure, completion of application may mean a state after spraying of a protective substance is performed so as to become a state where the protective substance is applied to the surface of the diffraction grating 152. Further, the completion of application may be defined as a state satisfying at least one of (i) a state in which a predetermined spray time of a protective substance has elapsed, (ii) a state in which a predetermined spray amount of a protective substance has been sprayed, and (iii) a state in which an application area is formed by a protective substance. However, a criterion for determining the completion of application may vary depending on embodiments and is not limited to the above example.

Furthermore, in the present specification, resumption of compression may mean transitioning to a state in which the laser pulse compressor 150 performs a compression operation of a laser pulse again. For example, the resumption of compression may be defined by at least one of (i) a time point at which a laser pulse is re-incident into the laser pulse compressor 150 and (ii) a time point at which an operation of compressing a laser pulse is initiated. However, a criterion for determining the resumption of compression may vary depending on embodiments and is not limited to the above example.

According to the laser pulse compressor of an embodiment of the present disclosure, a variation in a surface state of the diffraction grating 152 may be reduced during an operation process of the laser pulse compressor 150, and progress of surface contamination may be suppressed, mitigating a degradation in diffraction efficiency. Furthermore, a change in a surface state in an area including a laser irradiation area may be mitigated, and a deviation in a surface state of the diffraction grating 152 between operation cycles may be reduced. Furthermore, even when operating conditions vary, changes in the surface state between operation cycles can be reduced according to the setting of operating conditions related to spraying of the protective substance and irradiation of the removal light, thereby reducing variations in output characteristics of the laser pulse compressor 150.

Hereinafter, a method for protecting the diffraction grating 152 of the laser pulse compressor 150 performed by the laser pulse compressor 150 according to the embodiment of the present disclosure will be described.

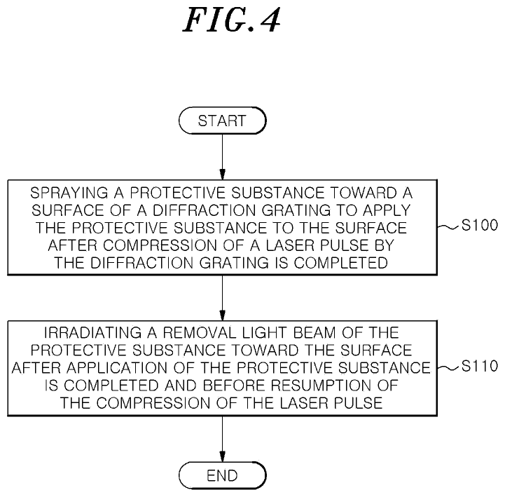

FIG. 4 is a flowchart illustrating a method for protecting the diffraction grating 152 of the laser pulse compressor 150 performed by the laser pulse compressor 150.

Here, a method for protecting the diffraction grating 152 according to the embodiment of the present disclosure may be performed by the above-described laser pulse compressor 150.

In addition, the flowchart illustrated in FIG. 4 is merely exemplary, and depending on embodiments, operations may be executed in a different order from that described in the flowchart, operations not described in the flowchart may be additionally executed, or one or more of the operations described in the flowchart may not be executed.

First, after compression of a laser pulse is completed, spraying a protective substance toward a surface of the diffraction grating 152 to apply the protective substance to the surface is performed (S100).

Completion of compression of a laser pulse may be implemented by generation or output of a laser pulse being stopped, or a laser pulse being blocked from being irradiated onto the diffraction grating 152 by a shutter disposed on a propagation path of the laser pulse. In this case, spraying of a protective substance may be initiated in response to a signal generated based on a driving state of the shutter.

The protective substance may include an organic material having a thiol group (—SH group).

Furthermore, in one embodiment, the protective substance may form a protective layer adsorbed to the surface by a chemical bond and blocking contact with contaminants.

Furthermore, applying the protective substance may include driving the nozzle 153 in response to a signal indicating completion of compression of a laser pulse.

Further, driving the nozzle 153 in response to the signal may include at least one of (i) generating a driving signal of the nozzle 153 from the signal indicating completion of compression and (ii) generating a driving signal of the nozzle 153 by detecting a change in the signal indicating completion of compression.

Furthermore, in one embodiment, at least one of a completion time of compression of a laser pulse and a spray time of a protective substance may include determining using a pre-trained artificial intelligence model.

Next, after application of a protective substance is completed and before resumption of compression of a laser pulse, irradiating a removal light beam of the protective substance toward a surface of the diffraction grating 152 is performed (S110).

A removal light beam may be irradiated under a set condition such that a protective substance applied to a surface of the diffraction grating 152 does not remain, and the removal light beam may include ultraviolet rays. Further, the light source 154 may be disposed to directly irradiate a removal light beam toward the surface of the diffraction grating 152, and an irradiation direction and an irradiation range of a light beam may be set to include an area where the applied protective substance is formed.

A removal light beam of a protective substance may include ultraviolet rays, and the ultraviolet rays may be irradiated in a continuous wave (CW) mode or a pulse mode. In the case of a continuous wave mode, a removal condition may be set based on at least one of an irradiation time length and an irradiation intensity, and in the case of a pulse mode, a removal condition may be set based on at least one of an accumulated irradiation amount (energy density) and the number of pulses. Here, an irradiation amount may be defined as a product of an irradiation intensity and an irradiation time in the case of continuous wave, and may be defined based on a product of energy per pulse and the number of pulses in the case of the pulsed light source 154.

An irradiation condition of removal ultraviolet rays may be set such that an irradiation intensity on the surface of the diffraction grating 152 becomes 10 to 50 mW/cm², or an accumulated irradiation amount becomes 1 to 10 J/cm², and an irradiation time length of the removal ultraviolet rays may be set to 10 to 60 minutes. Furthermore, in one embodiment, a wavelength of removal ultraviolet rays may include a 190 nm wavelength band.

Furthermore, an irradiation time length of a removal light beam of a protective substance may be determined depending on a time length of spraying the protective substance. For example, an operation rule in a form of a rule, a table, or a function may be applied such that an irradiation time length of a removal light beam is set long when a spray time of a protective substance is set long.

An irradiation time length of a removal light beam of a protective substance may be set longer as a spray time of the protective substance is longer. Setting of such an irradiation condition may change according to an operation condition.

Furthermore, in order to increase removal efficiency of a protective substance, a removal light beam may be irradiated in a state in which a vacuum level of the vacuum chamber 151 is adjusted. For example, after application of a protective substance is completed, a gate valve connected to the vacuum chamber 151 is closed to isolate the vacuum chamber 151 according to a type of the protective substance and an interaction mechanism with a removal light beam, a removal light beam is irradiated in a state in which a vacuum level is adjusted, and after removal of the protective substance is completed, irradiation of the removal light beam is stopped and the gate valve is opened to return the vacuum level of the vacuum chamber 151.

According to the method of the present disclosure, variations in the surface state of the diffraction grating 152 that may occur during a period from the end of operation to before resumption of operation of the laser pulse compressor 150 can be reduced, and the progression of surface contamination can be suppressed, thereby mitigating a decrease in diffraction efficiency. Furthermore, a deviation in a surface state of the diffraction grating 152 between operation cycles may be reduced, and accordingly, a variation in an output characteristic of the laser pulse compressor 150 can be reduced. In addition, even when a change in an operation condition exists, a variation in a spray timing can be reduced by determining a termination time or a spray time, reducing a change in a surface state between operation cycles.

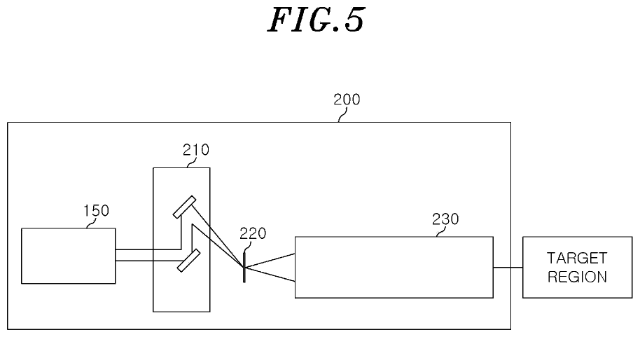

Hereinafter, a particle beam therapy apparatus 200 including the laser pulse compressor 150 will be described with reference to FIG. 5.

FIG. 5 is a schematic diagram illustrating a configuration of a particle beam therapy apparatus 200.

The particle beam therapy apparatus 200 may include a laser pulse compressor 150, a focusing optical system 210, a target 220, and a particle beam provider 230. The laser pulse compressor 150 herein may be the apparatus described above. Furthermore, the focusing optical system 210 may be disposed to focus a compressed laser pulse output from the laser pulse compressor 150 onto the target 220, and the target 220 may generate therapeutic particles by irradiation of the focused laser pulse. The particle beam provider 230 may be operable to provide a therapeutic particle beam using therapeutic particles generated by the target 220.

The laser pulse compressor 150 may output a compressed laser pulse.

The focusing optical system 210 may refer to an optical system including a lens, a mirror, or a combination thereof, and may be disposed to focus an output laser pulse onto the target 220.

The target 220 may mean a material or structure provided to generate therapeutic particles by irradiation of a laser pulse.

The particle beam provider 230 may include a configuration for transmitting, shaping, or setting an irradiation direction of generated therapeutic particles, and may be operable to provide a therapeutic particle beam to a target region (e.g., a body or a cell of a patient to be treated).

The target 220 may be a material or structure provided to generate therapeutic particles by irradiation of a laser pulse, and may further include a particle beam provider 230 providing a therapeutic particle beam using the therapeutic particles generated by the target 220.

The target 220 may include tungsten, and particles generated by the target 220 may include X-rays.

According to the embodiment of the present disclosure as described above, a therapeutic particle beam may be provided using a compressed laser pulse output from the laser pulse compressor 150, and when an output characteristic variation between operation cycles of the laser pulse compressor 150 is reduced, operational stability of providing a therapeutic particle beam may be improved.

The above-described embodiments may be implemented by various means. For example, each block in the above-described block diagrams and each operation in the flowcharts may be implemented by hardware, firmware, software, or a combination thereof, and may be performed by the processor 157 executing at least one instruction stored in the memory 156.

Furthermore, the above-described embodiments may be implemented in a form including instructions stored in a computer-readable recording medium.

Meanwhile, an execution sequence of the operations illustrated in the drawings is exemplary, and depending on embodiments, some operations may be performed in parallel, the sequence may be changed, or some operations may be added or omitted.

The above description is merely illustrative of the technical idea of the present disclosure, and those of ordinary skill in the art to which the present disclosure pertains may make various modifications and variations without departing from an essential quality of the present disclosure. Therefore, the embodiments disclosed in the present disclosure are not intended to limit the technical idea of the present disclosure, but to describe, and the scope of the technical idea of the present disclosure is not limited by these embodiments. The protection scope of the present disclosure should be interpreted by the claims below, and all technical ideas within the equivalent scope should be interpreted as being included in the scope of rights of the present disclosure.

1. A laser pulse compressor comprising:
a diffraction grating for compressing an incident laser pulse;
a nozzle spraying a protective substance such that the protective substance is applied onto a surface of the diffraction grating after compression of each laser pulse group is completed; and
a light source configured to irradiate a removal light beam for removing the protective substance toward the surface of the diffraction grating after application of the protective substance is completed and before resumption of compression of a subsequent laser pulse group.

2. The laser pulse compressor of claim 1, wherein the surface is activated to a predetermined energy state after completion of the compression of each laser pulse group, and the protective substance is applied onto the surface of the diffraction grating in an activated state.

3. The laser pulse compressor of claim 1, wherein the protective substance includes an organic material having a thiol group.

4. The laser pulse compressor of claim 1, wherein the protective substance forms a protective layer adhered to the surface of the diffraction grating by a chemical bond to block contact with contaminants.

5. The laser pulse compressor of claim 1, wherein the nozzle is disposed such that a spray direction of the protective substance directs an area onto which the laser pulse is irradiated among the surface of the diffraction grating.

6. The laser pulse compressor of claim 5, wherein the nozzle is disposed such that an irradiation area of the laser pulse is 80% or less of an area where the protective substance is applied.

7. The laser pulse compressor of claim 1, wherein the nozzle is driven in response to a signal indicating completion of the compression of each laser pulse group.

8. The laser pulse compressor of claim 7, wherein the nozzle is driven by a predetermined driving signal, and the driving signal is generated using the signal indicating the completion of the compression.

9. The laser pulse compressor of claim 1, wherein a spray start time of the protective substance from the nozzle is determined based on a time length during which each laser pulse group is irradiated onto the diffraction grating.

10. The laser pulse compressor of claim 9, wherein the spray start time of the nozzle is advanced as an irradiation time of each laser pulse group increases.

11. The laser pulse compressor of claim 1, wherein an irradiation time length of the removal light beam of the protective substance is determined depending on a time length of spraying the protective substance.

12. The laser pulse compressor of claim 11, wherein as the spray time of the protective substance increases, the irradiation time length of the removal light beam for removing the protective substance increases.

13. The laser pulse compressor of claim 1, wherein at least one of a completion time of the compression of each laser pulse group and a spray time of the protective substance is determined based on a pre-trained artificial intelligence model.

14. A method for protecting a diffraction grating of a laser pulse compressor performed by the laser pulse compressor, the laser pulse compressor including the diffraction grating, the method comprising:
spraying a protective substance toward a surface of the diffraction grating to apply the protective substance to the surface of the diffraction grating after compression of each laser pulse group by the diffraction grating is completed; and
irradiating a removal light beam for removing the protective substance toward the surface of the diffraction grating after application of the protective substance is completed and before resumption of the compression of a subsequent laser pulse group.

15. The method of claim 14, wherein the protective substance includes an organic material having a thiol group.

16. The method of claim 14, wherein the protective substance forms a protective layer adhered to the surface of the diffraction grating by a chemical bond to block contact with contaminants.

17. The method of claim 14, wherein the laser pulse compressor includes a nozzle, and
wherein the method further comprises:
driving the nozzle in response to a signal indicating completion of the compression of each laser pulse group.

18. The method of claim 17, wherein the nozzle is driven by a driving signal generated from the signal indicating the completion of the compression.

19. The method of claim 14, wherein at least one of a completion time of the compression of each laser pulse group and a spray time of the protective substance is determined based on a pre-trained artificial intelligence model.

20. A particle beam therapy apparatus comprising:
the laser pulse compressor of claim 1;
a focusing optical system focusing a compressed laser pulse output from the laser pulse compressor;
a target generating therapeutic particles using the compressed laser pulse focused by the focusing optical system; and
a particle beam provider configured to provide a therapeutic particle beam using the therapeutic particles generated by the target.
